(19) United States
(12) Patent Application Publication
(10) Pub. No.: US 2006/0218989 A1
(43) Pub. Date: Oct. 5, 2006

(54) Method and apparatus for monitoring catalytic abator efficiency

(76) Inventors: Dominic Cianciarelli, Nepean (CA); Jorge Marson, Nepean (CA)

Correspondence Address: IAN FINCHAM, SUITE 606, 225 METCALFE STREET, OTTAWA, ON, K2P 1P9, CA

(21) Appl. No.: 11/388,622

(22) Filed: Mar. 24, 2006

(30) Foreign Application Priority Data
Mar. 30, 2005 (WO) PCT/CA05/00469

(51) Int. Cl. G01N 33/497

(52) U.S. Cl. 73/23.31

(57) ABSTRACT

The present invention provides for a method and apparatus to measure exhaust stream emissions of a catalytic abator for determining the efficiency of the removal of a treatment gas including the steps of providing a calibrated sensor to measure the selected range of the treatment gas concentration in the abator from an exhaust stream after the treatment gas has been treated to verify abator efficiency and output or control the abator based on the efficiency values generated.

### FIELD OF THE INVENTION

The present invention relates to a gas treatment process wherein the residual treatment gas is vented to the atmosphere. More specifically, there is provided a method and apparatus for monitoring emissions of a catalytic abator to determine the efficiency of the abator.

### BACKGROUND OF INVENTION

A catalytic abator is used to convert airborne volatile organic compounds such as ethylene oxide into harmless carbon dioxide and water vapor. The abator offers controlled purification with a high destruction efficiency for use in sterilization processes.

Sterilizers commonly employ a combination of dichlorodifluoromethane (CFC-12) and ethylene oxide (EtO). EtO is the actual sterilant whereas CFC-12 is used as a diluent to form a non-flammable blend. A typical combination is 12% EtO mixed with 88% CFC-12, referred to as “12/88”. The CFC-12 is a Class I ozone depleting substance and has been phased out and replaced by a 100% EtO sterilant or a blend of EtO and carbon dioxide. EtO is a toxic air pollutant and is under proposal for federal regulation. Used EtO is fed into a heated air stream where it is diluted and catalytically converted into carbon dioxide and water vapor to be released into the ambient air. Of course, abator efficiency is crucial given its potential to release a human carcinogen.

Sterlizers, or abators, are commonly used, particularly in health care facilities or any field in which products are routinely sterilized, and generally includes an enclosed catalyst bed, an air heater, a fan and the controls necessary to complete the operation.

The conventionally known techniques for measuring abator efficiency are typically costly and/or lengthy processes. For example, one method involves the determination of the mass of compound at both the inlet and the outlet of the control equipment. The mass at the inlet may be either estimated or determined from an actual measurement of the flow and concentration at the inlet to the abator while the outlet mass is determined by measuring the flow and concentration of the exhaust at the outlet. Several drawbacks to this methodology include the potential for the sample at the inlet to be dangerous and the handling can expose the sampling personnel to one or more toxic compounds. For example, EtO at room temperature is flammable and potentially explosive. Further, the samples must be collected in bags and must be analyzed either at an on-site lab or, more typically, at an external lab within a specified period of time causing the potential for condensation during the collection of the samples at the outlet resulting in EtO losses. Obviously, the losses due to condensation and prolonged storage of the bag samples prior to analysis will result in the overestimation of the control efficiency. In addition, the known methods also require that the collection of the bags be timed precisely to include large concentrations at-the beginning of the abator cycle. A failure to include the large concentrations can result in inaccuracies in the data collected.

Consequently, there is a need for a method and apparatus to achieve a safer, instantaneous, cheaper and more accurate determination and control of the efficiency of the abator which is independent of the inlet mass to the abator, the dilution and excess air in the system, and which can be programmed to determine the control efficiency for any portion of the cycle.

### SUMMARY OF THE INVENTION

The present invention provides for a method for measuring exhaust stream emissions of a catalytic abator for determining the efficiency of the removal of a treatment gas, the steps comprising:
a) providing a calibrated sensor to measure a selected range of the treatment gas concentration;
b) providing an exhaust stream from the abator after the treatment gas has been treated by the abator;
c) withdrawing at least a portion of the exhaust stream from the abator and passing the withdrawn portion of the exhaust stream into the sensor;
d) measuring the concentration of treatment gas in the withdrawn portion of the exhaust stream in the selected range; and,
e) verifying the abator efficiency based on the measurement.

Preferably, there is provided the step of diluting the portion of an exhaust stream with cleaned and dried ambient air, utilizing an exhaust fan in the abator to mix the exhaust from a sterilizer with ambient air, controlling the amount of the exhaust stream withdrawn from the abator exhaust and subjecting the withdrawn exhaust to a cleaning step after sensing has been completed.

Further, it is preferred there is provided the step of controlling the flow of the withdrawn exhaust stream to the sensor and the sensing step includes the steps of sensing at least one of carbon dioxide or ethylene oxide.

It is also desirable the sensor is a photo acoustic sensor, an electrochemical sensor or a non-dispersive infrared sensor, the withdrawn portion of the exhaust stream from the abator is dehumidified and the abator is automatically adjusted for optimum efficiency based on the efficiency measurements.

In another embodiment of the present invention there is provided a device for monitoring the efficiency of a sterilizer treatment gas abator, comprising:
a monitoring device having an inlet for receiving a treatment gas from an outlet of the abator and an outlet, the treatment gas having been treated by the abator to remove the treatment gas;
the monitoring device including a calibrated treatment gas sensor and means for withdrawing at least a portion of the exhausted gas stream from the abator outlet;
the sensor being positioned and adapted to measure the treatment gas in the withdrawn stream to permit verification of the efficiency of the abator; and
output means for verifying the efficiency measurements.

The abator preferably includes an exhaust fan and a catalyst together with a means or heating the catalyst, the monitoring device includes control means for controlling the amount of exhaust withdrawn from the abator and the monitoring device includes cleaning means for cleaning the withdrawn exhaust after sensing has been completed.

It is also preferable the monitoring device includes at least one flow controller for controlling the flow of gas, the sensor includes a function for sensing at least one of carbon dioxide or ethylene oxide, the sensor is a photo acoustic sensor, an electrochemical sensor or a non-dispersive infrared sensor and the abator includes dilution means for diluting the withdrawn gas.

Moreover, it is desirable the abator includes cleaning means, the abator includes drying means for dehumidifying the treatment gas, the monitoring device includes a monitor for displaying the efficiency values and the efficiency of the abator is adjusted automatically based on said efficiency measurements.

From the above, it is found that the present invention allows the real-time determination of the abator control efficiency that can be used to provide real-time continuous control of the abator operation. Further, the feedback to the abator control module can be used to optimize the heat input (electrical heater) and throttling pattern of the feed from the sterilizer chamber to optimize the control efficiency of the abator on a continuous basis.

### BRIEF DESCRIPTION OF THE DRAWINGS

Having generally defined the invention reference will now be made to the accompanying drawings with respect to the preferred embodiments.

FIG. 1 shows a schematic view of a sterilization process including an abator in use with an efficiency monitoring apparatus of the present invention;

FIG. 2 shows a schematic view of the abator and monitoring device of FIG. 1;

FIG. 3 shows the theoretical effect of dilution air;

FIG. 4 shows two graphs illustrating the relationship between the concentrations of CO₂/EtO and the abator efficiency; and,

FIG. 5 shows the effect of dilution on the determination of control efficiencies.

### DETAILED DESCRIPTION OF THE DRAWINGS

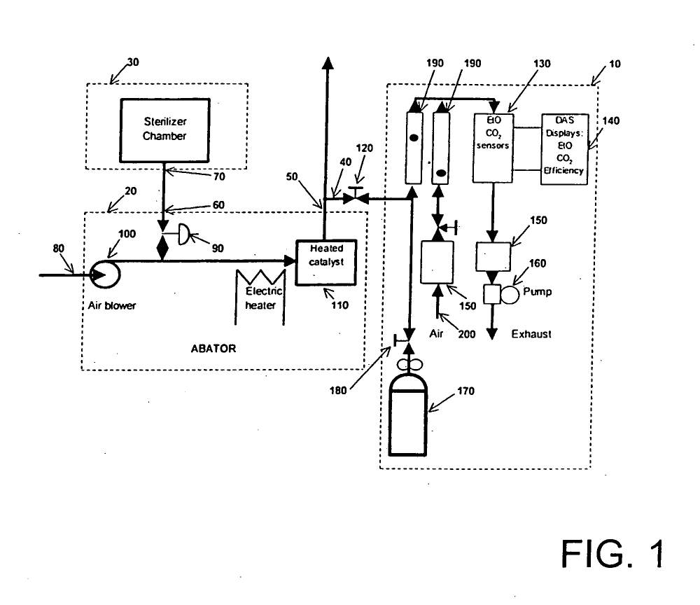

Referring now to FIG. 1, there is illustrated a system for carrying out a method of monitoring a gas treatment process. The system includes a monitoring apparatus 10 which is provided for use with an abator 20 and a sterilizer 30. The monitoring apparatus 10 is in fluid communication with the abator 20 by way of conduit line 40 and an outlet 50 of the abator 20. The abator 20 has an inlet 60 in fluid communication with the sterilizer chamber 30 by way of outlet 70 from which the sterilizer 30 exhausts the treatment gas.

The sterilizer 30 is of the conventional type typically used in hospitals, research facilities and other similar institutions requiring sterilization processes using potentially hazardous treatment gases. Similarly, the abator 20 is of the conventional type and typically includes elements common to such devices such as a dilution inlet 80, an exhaust fan 90, a throttling valve 100 and a heated catalyst 110, all of which are illustrated for the purposes of example in FIG. 1. However, the common elements outlined above and as shown in FIG. 1 are not intended to be limiting as any suitable abator device can be readily substituted as would be understood by those skilled in the art.

Monitoring apparatus 10 includes a control means 120 to control the amount of exhaust sampled from the abator 20, a sensing means 130 for measuring trace components in the exhaust and output means 140 for output of the control efficiency results. An air cleaning means 150 suitable for cleaning the sampled exhaust, after sensing has been completed, is provided to release the exhaust into the ambient air is also provided along with a pump 160 for drawing the sampled exhaust into the monitoring apparatus 10.

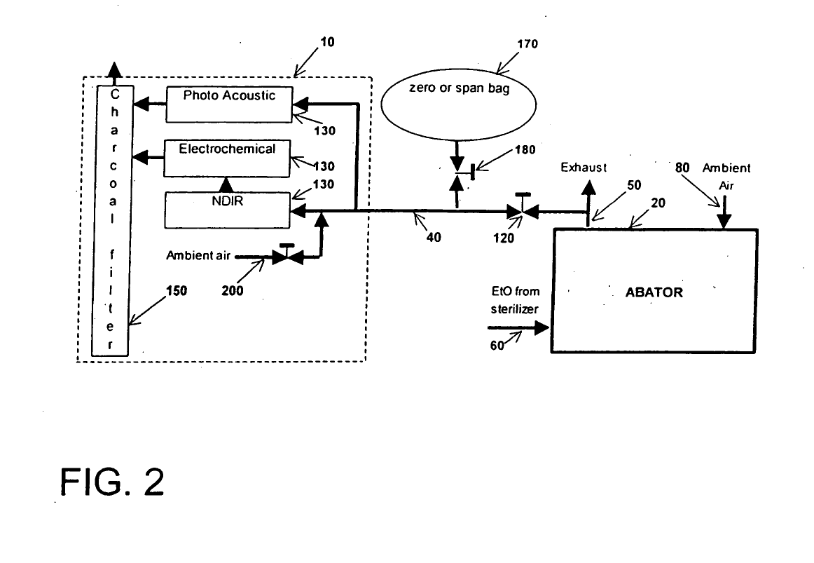

Referring to FIG. 1 and FIG. 2, the sensing means 130 will be calibrated prior to operation of the apparatus to ensure accurate quantification of the treatment gas. A calibration means is provided and can include a source 170 of the treatment gas and a source control means 180 such as a valve. The source 170 can be any conventional method such as a zero or span gas bag. Obviously, the type of gas used in the source 170 for completing the calibration of the sensor is similar to that used in the sterilization process. The gas passes into the monitoring apparatus 10 through conduit 40 and through one or more flow controllers 190 (for example a rotameter) before entering the sensing means 130 to make the necessary adjustments.

Although the calibration means set out above is preferable, it would be readily understood that any conventionally known method of calibrating the sensing means 130 can be used. Once the sensing means 130 has been appropriately calibrated the monitoring apparatus may then be placed into operation.

During operation of the present invention, an exhaust of a gas or fluid having a gas to be treated and having exited the sterilizer chamber 30 by way of the outlet 70 enters the abator 20 via inlet 60. In turn, the treated gas in abator 20 is exhausted by way of outlet 50. To effect monitoring of the abator 20, a portion of the exhaust gas or fluid is drawn from outlet 50 in a controlled manner by way of, for example, a valve 120. As the exhaust portion is being drawn into the monitoring apparatus 10 and controlled throughout by suitable control means 190, before subsequently entering sensing means 130 for sensing traces of treatment gas components present in the exhaust from abator 20.

It is to be understood that the sensing means 130 can include one or more sensing devices for sensing the trace treatment gas as well as other gases such as, for example CO₂ to carry out the analysis of the reading. Further, the sensing means 100 can operate singularly or in parallel as shown in FIG. 2 depending on the required application. For example, the different types of sensors contemplated by the present invention include but are not limited to photo acoustic devices, electrochemical devices and non-dispersive infrared devices, etc.

It has been found that some environments having a high humidity value introduce condensation in the apparatus 10 that results in inaccurate readings of the treatment gas. As the exhaust typically includes trace amounts of the treatment gas which are effected by condensation there is optionally to be provided a dilution means 200 for diluting the withdrawn sample with ambient air. The dilution means 200 can also include a cleaning means 150 to remove unwanted impurities from the ambient air. The cleaning means 150 can further include a dryer (not shown) to further inhibit condensation of the treatment gas prior to entry in the sensing means 130.

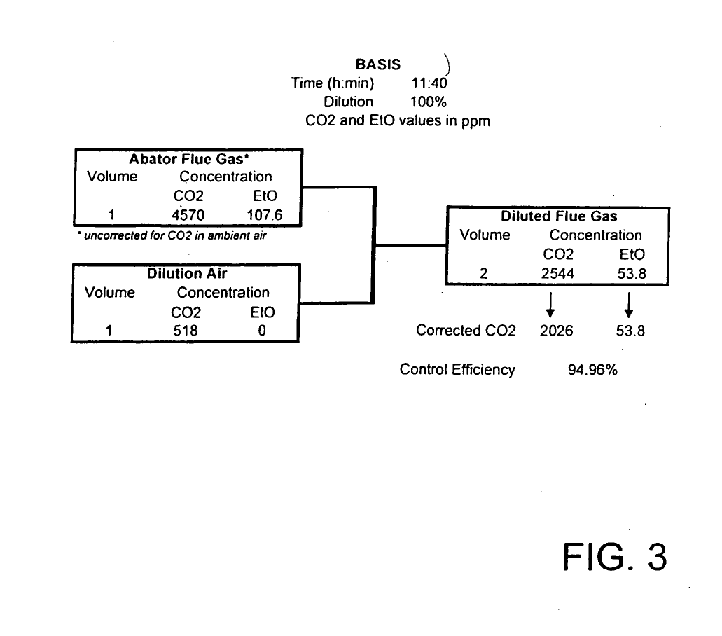

It is important to note that the determination of control efficiency is independent of the dilution of the exhaust gases. A theoretical example using the readings at 11:40 from Table 1 is shown in FIG. 3. In the example, as both the CO₂ and EtO are diluted by the same amount, there is no change in the control efficiency. This is applicable for all dilution ratios. Of course, it is readily understood that excessive dilution will render the target concentration below the sensitivity of the instrument and is outside the parameters of the present invention.

The results of the detection of the treatment gas by sensing means 130 having been sensed and calculated by calculation means can then be output by a suitable output means 140. The output means can include a monitor to display results for verification purposes or manual adjustments. This invention also contemplates the use of automatic adjustments of the abator 20, through suitable means for the detected treatment gas to remain below the predetermined range.

The calculation means (not shown) provides an output of control efficiency determined by using the following unique relationship which utilizes the carbon dioxide in the flue gas as a surrogate for the destroyed gas, for example ethylene oxide. The amount of carbon dioxide formed by way of this example follows a stoichiometric quantity based on the equation below. The concurrent measurement of CO₂ and EtO provides both the inlet and outlet components for the control efficiency calculation. The stoichiometric relationship and the determination of control efficiency are detailed below.

$${C}_{2} {H}_{4} O + 2.5 ( {O}_{2} + 3.78 {N}_{2} ) \to 2 {CO}_{2} + 2 {H}_{2} O + 2.5 ( 3.78 {N}_{2} )$$

$$Control Efficiency = \frac{{C}_{{CO}_{2}}}{{C}_{{CO}_{2}} + 2 {C}_{EtO}} for pure EtO sterilizers$$

$$where EtO and {CO}_{2} are measured at the outlet$$

$$or$$

$$Control Efficiency = \frac{{C}_{{CO}_{2}} - \frac{1 - y}{y} {C}_{EtO}}{{C}_{{CO}_{2}} + 2 {C}_{EtO}} for EtO / {CO}_{2} mixtures$$

$$where y is the volume fraction of EtO in the sterilization mixture$$

This relationship between the above calculation and the method and apparatus of the present invention is now shown by way of Example 1.

### EXAMPLE 1

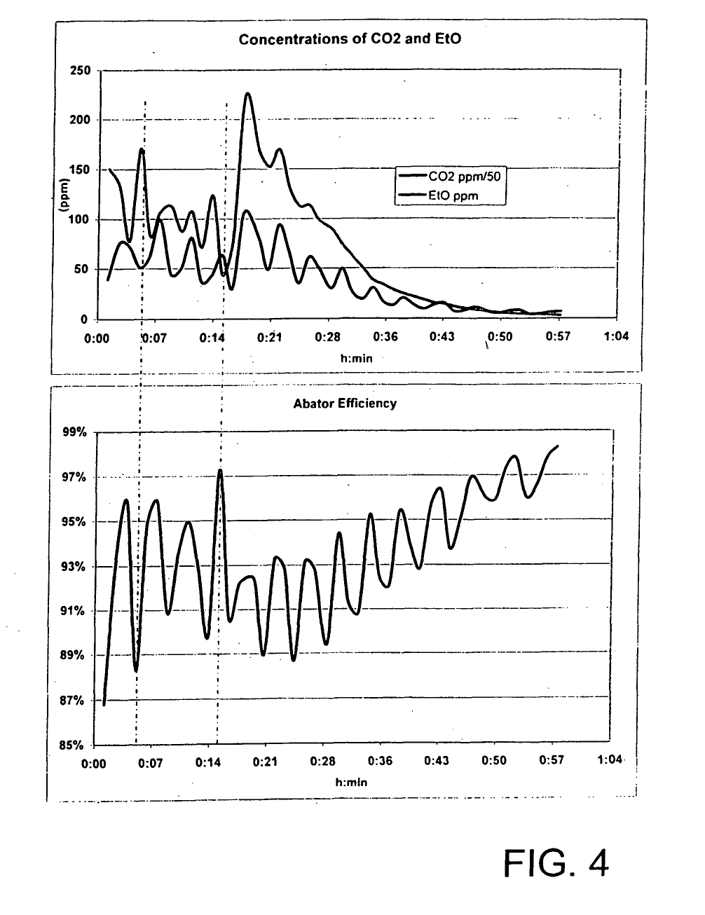

The determination of control efficiencies are given in both FIG. 4 and in Table 1 below. Table 1 illustrates data collected at the exhaust of a Donaldson abator treating sterilizer gases from a 3M sterilizer. The flue gas sample was analyzed with a Bruel & Kjaer Model 1302 analyzer which uses the photoacoustical (PA) detection of infra red active gases such as EtO, CO₂ and H₂O. This instrument operates on measurement cycles of slightly greater than one (1) minute. Prior to the field testing, the instrument was calibrated with separate gas standards of each compound, and the analyzer created response corrections for the compounds of interest, so as to minimize cross sensitivity.

The abator (catalytic oxidizer) requires a large volume of ambient air for the catalytic oxidation of EtO that is far in excess of that required for stoichiometric oxidation. One reason for the additional volume is to keep the catalyst in the abator from overheating. The flow from the sterilizer chamber represents a minor fraction (approximately one percent) of the total flow exiting the abator.

As ambient air contains carbon dioxide, the concentrations of carbon dioxide from the PA analyzer are corrected for this background level prior to calculating the control efficiency.

As shown by way of example in the data in Table 1 below, the concentrations of CO₂ and EtO are cyclical. Traces of the exhaust components exhibit a sinusoidal pattern with a frequency of four (4) minutes. This pattern is independent of the levels of CO₂ and EtO. On the basis of the efficiency, the increasing variations of the abator efficiency are measured in the first 16 minutes of the abator cycle. This is followed by decreasing variations with an upward trend in efficiency towards the end of the cycle.

Note that the one-hour cycle shown in FIG. 4 corresponds to the evacuation phase of the sterilizer chamber. This is followed by a much longer period of aeration of the sterilizer chamber. Opposite trends in the concentration of CO₂ and EtO denote either increases or decreases in efficiency. Two examples are noted in FIG. 4 with vertical dashed lines. In the case of the first dashed line (at 05:11, left line), the EtO concentration increased and the CO₂ dropped. This corresponds to a drop in efficiency. On the other hand, a rise in CO₂ and corresponding drop in EtO denotes an increase in efficiency as is evidenced in the second dashed line (at 15:29, right line). The majority of the EtO releases notably occur in the first hour of the abator cycle.

TABLE 1
Temporal Variation of Efficiency Using Ethylene Oxide and
Carbon Dioxide Concentrations Using a Photoacoustic Analyzer

| Time | CO₂* | EtO | Efficiency |
|---|---|---|---|
| (min:s) | (ppm) | (ppm) | (%) |
| 01:15 | 1982 | 150.6 | 86.81 |
| 02:38 | 3792 | 132.6 | 93.46 |
| 03:54 | 3612 | 78.4 | 95.84 |
| 05:11 | 2572 | 170.6 | 88.29 |
| 06:26 | 3092 | 84.5 | 94.82 |
| 07:41 | 4982 | 106.6 | 95.90 |
| 09:07 | 2242 | 112.6 | 90.87 |
| 10:23 | 2512 | 87.6 | 93.48 |
| 11:40 | 4052 | 107.6 | 94.96 |
| 12:56 | 1862 | 71.9 | 92.83 |
| 14:13 | 2182 | 123.6 | 89.83 |
| 15:29 | 3182 | 44.2 | 97.30 |
| 16:48 | 1512 | 78.0 | 90.65 |
| 18:09 | 5322 | 224.6 | 92.22 |
| 19:59 | 4062 | 167.6 | 92.38 |
| 21:14 | 2452 | 152.6 | 88.93 |
| 22:30 | 4692 | 169.6 | 93.26 |
| 23:46 | 3392 | 131.6 | 92.80 |
| 25:02 | 1762 | 112.6 | 88.67 |
| 26:17 | 3082 | 113.6 | 93.14 |
| 27:33 | 2522 | 98.3 | 92.77 |
| 29:08 | 1502 | 89.1 | 89.40 |
| 30:27 | 2482 | 73.9 | 94.38 |
| 31:45 | 1302 | 61.8 | 91.33 |
| 33:04 | 992 | 50.0 | 90.85 |
| 34:20 | 1532 | 38.3 | 95.24 |
| 35:38 | 832 | 33.9 | 92.47 |
| 36:54 | 662 | 28.6 | 92.05 |
| 38:09 | 1042 | 25.0 | 95.42 |
| 39:37 | 642 | 21.2 | 93.81 |
| 40:52 | 482 | 18.5 | 92.88 |
| 42:08 | 692 | 15.5 | 95.72 |
| 43:23 | 762 | 14.4 | 96.36 |
| 44:38 | 355 | 11.9 | 93.73 |
| 45:54 | 392 | 10.1 | 95.11 |
| 47:10 | 562 | 8.8 | 96.95 |
| 48:57 | 320 | 6.7 | 95.99 |
| 50:13 | 273 | 5.8 | 95.96 |
| 51:30 | 380 | 5.1 | 97.37 |
| 52:47 | 409 | 4.7 | 97.78 |
| 54:02 | 205 | 4.2 | 96.04 |
| 55:17 | 230 | 4.2 | 96.52 |
| 56:34 | 296 | 3.4 | 97.79 |
| 57:49 | 341 | 3.0 | 98.26 |

| | |
|---|---|
| Average | 93.65 |

*CO2 in ambient air (518 ppm) correction included for each value

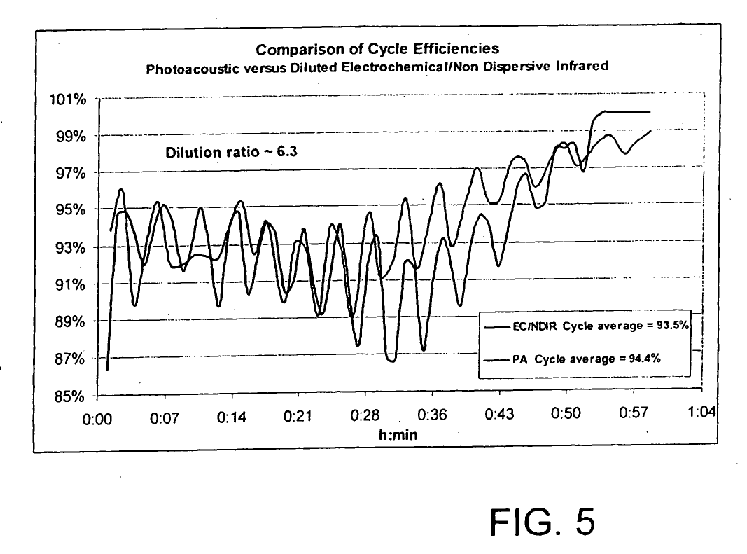

An example of an actual dilution on the exhaust of an abator is illustrated in FIG. 5. The example provides for two types of instruments used in parallel for the measurement of CO₂ and EtO. A PA instrument was used for the undiluted sample whereas paired sensors using electrochemical sensing (EC for EtO) and non dispersive infra red (NDIR for CO₂) were utilized for the diluted abator exhaust. Air was used to dilute the exhaust gas with the dilution ratio being estimated based on the concurrent levels of CO₂ from the PA and NDIR. For this example the dilution ratio was estimated at 6.3 times.

The two types of sensing technologies (PA versus EC/NDIR) used were different in terms of sampling frequency and response characteristics. The difference is reflected in the efficiency values for both methods reflected in the whole hour and is particularly true for the second half of the cycle. The difference in the efficiencies in the second half (right side) of the cycle are a reflection of the two sampling technologies rather than the control efficiency determination technique. The shallow hump for the PA analyzer at 11 or 12 minutes was due to a skipped analysis cycle on the instrument.

FIG. 5 is illustrative that despite the two measurement technologies being used, and the wide range of CO₂ and EtO concentrations between the PA and Electrochemical/ non-dispersive infra red levels (EC/NDIR), the effect of dilution did not preclude the calculation of control efficiency using the method in this application. Both traces showed the same pattern. Efficiencies decreased towards the middle of the cycle and showed an increasing trend to full efficiency at the end of the hour. The overall control efficiencies were 93.5 and 94.4% for the EC/NDIR and PA techniques respectively, a difference of one percent.

On-line Monitoring Versus Integrated Sampling

The reliability of an on-line method to represent an abator cycle was addressed by collecting an integrated sample of the abator exhaust gases (not shown) for the one hour evacuation period. In this method, a sample is continuously drawn from the exhaust flue at a constant rate and stored in an enclosed container such as a flexible bag or canister. The integrated sample, containing a representative portion of the exhaust gases, was analyzed using the same instrumentation as the on-line measurement. This approach minimizes any difference in response due to different sensing methods.

The results shown in Table 2 indicate that the average of the periodic efficiency readings (in this case, every minute) is identical to a single integrated efficiency over the sample period.

TABLE 2
Comparison of On-line Monitoring Versus an
Integrated Sample Using a Photoacoustic Analyzer

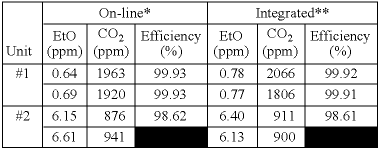

*EtO and CO₂ readings represent an average of 45 to 50 readings. Efficiency based on average of 45 to 50 efficiencies.
**Integrated sample collected in a 6-Litre pacivated stainless steel canister. Efficiency based on a single EtO and CO₂ reading.

The result of the application of an on-line method being an accurate indication of overall cycle efficiency.

While the above relates to sterilizer abatement devices, it is understood to those skilled in the art the above method and apparatus may be adapted to other systems having a gas treatment process wherein the residual treatment gas is vented to the atmosphere. For example, this system may be applied to control equipment with a well-defined inlet gas and catalytic oxidizer with unique products of combustion such as hydrocarbons (carbon tetrachloride, Freons, volatile organic compounds (VOC's), acid gases and carbon dioxide etc.)

The above clearly provides for a monitoring of catalytic abator efficiency in a safer, instantaneous and more efficient manner than previously known methodologies.

1. A method for measuring exhaust stream emissions of a catalytic abator for determining the efficiency of the removal of a treatment gas, the steps comprising:
a) providing a calibrated sensor to measure a selected range of the treatment gas concentration;
b) providing an exhaust stream from the abator after the treatment gas has been treated by the abator;
c) withdrawing at least a portion of the exhaust stream from the abator and passing the withdrawn portion of the exhaust stream into the sensor;
d) measuring the concentration of treatment gas in the withdrawn portion of the exhaust stream in the selected range; and,
e) verifying the abator efficiency based on the measurement.

2. The method of claim 1, further comprising the step of diluting the portion of an exhaust stream with cleaned and dried ambient air.

3. The method of claim 1 or 2, further including the step of utilizing an exhaust fan in the abator to mix the exhaust from a sterilizer with ambient air.

4. The method of any one of claims 1 to 3, further including the step of controlling the amount of the exhaust stream withdrawn from the abator exhaust.

5. The method of any one of claims 1 to 4, further including the step of subjecting the withdrawn exhaust to a cleaning step after sensing has been completed.

6. The method of any one of claims 1 to 5, further comprising the step of controlling the flow of the withdrawn exhaust stream to the sensor.

7. The method of any one of claims 1 to 6, wherein the method includes the steps of sensing at least one of carbon dioxide or ethylene oxide.

8. The method of any one of claims 1 to 7, wherein the sensor is a photo acoustic sensor, an electrochemical sensor or a non-dispersive infrared sensor.

9. The method of any one of claims 1 to 8, wherein the method includes the step of dehumidifying the withdrawn portion of the exhaust stream from the abator.

10. The method of any one of claims 1 to 9, wherein the abator is automatically adjusted for optimum efficiency based on the efficiency measurements.

11. A device for monitoring the efficiency of a sterilizer treatment gas abator, comprising:
a monitoring device having an inlet for receiving a treatment gas from an outlet of the abator and an outlet, the treatment gas having been treated by the abator to remove the treatment gas;
the monitoring device including a calibrated treatment gas sensor and means for withdrawing at least a portion of the exhausted gas stream from the abator outlet;
the sensor being positioned and adapted to measure the treatment gas in the withdrawn stream to permit verification of the efficiency of the abator; and
output means for verifying the efficiency measurements.

12. The device of claim 11, wherein the abator includes an exhaust fan and a catalyst together with a means for heating the catalyst.

13. The device of claim 11 or 12, wherein the monitoring device includes control means for controlling the amount of exhaust withdrawn from the abator.

14. The device of any one of claims 11 to 13, wherein the monitoring device includes cleaning means for cleaning the withdrawn exhaust after sensing has been completed.

15. The device of any one of claims 11 to 14, wherein the monitoring device includes at least one flow controller for controlling the flow of gas.

16. The device of any one of claims 11 to 15, wherein the sensor includes a function for sensing at least one of carbon dioxide or ethylene oxide.

17. The device of any one of claims 11 to 16, wherein the sensor is a photo acoustic sensor, an electrochemical sensor or a non-dispersive infrared sensor.

18. The device of any one of claims 11 to 17, wherein the abator includes dilution means for diluting the withdrawn gas.

19. The device of any one of claims 11 to 18, wherein the abator includes cleaning means.

20. The device of any one of claims 11 to 19, wherein the abator includes drying means for dehumidifying the treatment gas.

21. The device of any one of claims 11 to 20, wherein the monitoring device includes a monitor for displaying the efficiency values.

22. The device of any one of claims 11 to 21, wherein the efficiency of the abator is adjusted automatically based on the efficiency measurements.
